(19) United States
(12) Patent Application Publication
(10) Pub. No.: US 2011/0076339 A1
(43) Pub. Date: Mar. 31, 2011

(54) Colon Cleansing Method and Kit

(75) Inventors: Stephen J. Vanner, Kingston (CA); Lawrence C. Hookey, Kingston (CA)

(73) Assignee: Kingston General Hospital, Kingston, ON (CA)

(21) Appl. No.: 12/994,102

(22) PCT Filed: May 22, 2009
(86) PCT No.: PCT/CA2009/000695

§ 371 Date: Nov. 22, 2010

Related U.S. Application Data
(60) Provisional application No. 61/055,811, filed on May 23, 2008.

(51) Int. Cl. A61K 33/42; A61K 36/48; A61K 31/4402; A61K 31/194; A61P 1/10

(52) U.S. Cl. 424/605; 424/757; 514/277; 514/574

(57) ABSTRACT

A method of colon cleansing that provides superior cleansing. In an embodiment, superior cleansing of the right colon is provided. In an embodiment, a stimulant laxative is consumed at least two days prior to the day for which maximum cleansing is desired, and an osmotic purgative is consumed one day prior to the day for which maximum cleansing is desired. A regimen that administered PICO-SALAX® plus BISACODYL™ was superior in cleansing the right colon compared to PICO-SALAX® alone, and when compared to Oral Sodium Phosphate. Patient tolerability and safety were excellent.

### FIELD OF THE INVENTION

The invention relates to a method for colonic cleansing. The invention also relates to a method of colonic cleansing that is effective for cleaning the whole colon which includes the ascending (right side) colon. The invention further relates to a kit for colonic cleansing.

### BACKGROUND OF THE INVENTION

to Investigations of the lining of the colon are performed to check for abnormalities such as, for example, gastrointestinal malignancies, inflammation, bleeding, and/or polyps, which may be associated with gastrointestinal disorders such as colon cancer, pre-malignant polyps, diarrhea, and inflammatory bowel disease. A colonoscopy is a visual inspection of the colon lining and is performed using an endoscope, which is a long tube with a video camera and light on its end.

Colon screening programs have been implemented worldwide resulting in a dramatic increase in the amount of colonoscopies performed. This continuing increase is straining existing endoscopic facilities and staff.

Pre-colonoscopy colon cleansing is necessary to enhance the detection rate of polyps. This is particularly the case for detection of flat-type polyps which are difficult to visualize since they protrude only a short distance from the colon lining plane. Compounding the difficulty with visualization due to their shape, flat-type colorectal cancers occur preferentially in the ascending colon (Okamoto et al., 2005), which is the most difficult area to prepare for endoscopy since it is prone to film and mucous coatings which obscure visual inspections. Hence, an improved pre-colonoscopy colon cleansing regimen would provide superior cleansing in the right colon or have excellent patient tolerance and safety, or both.

Despite the availability of several different products for colon cleansing, there continues to be a need for a better regimen which provides excellent colon cleansing without tolerability and safety concerns.

### SUMMARY OF THE INVENTION

An aspect of the invention provides a method of colon cleansing comprising consuming an effective amount of a stimulant laxative 3 days and 2 days prior to the day for which maximum cleansing is desired; drinking fluids and restricting solid food for one day prior to the day for which maximum cleansing is desired; and consuming an effective amount of an osmotic purgative one day prior to the day for which maximum cleansing is desired.

Another aspect of the invention provides a method of colon cleansing comprising consuming an effective amount of a stimulant laxative at least 2 days prior to the day for which maximum cleansing is desired; drinking fluids and is restricting solid food for one day prior to the day for which maximum cleansing is desired; and consuming an effective amount of an osmotic purgative one day prior to the day for which maximum cleansing is desired.

Yet another aspect of the invention provides a method of colon cleansing comprising consuming an effective amount of a stimulant laxative 4, 3, and 2 days prior to the day for which maximum cleansing is desired; drinking fluids and restricting solid food for one day prior to the day for which maximum cleansing is desired; and consuming an effective amount of an osmotic purgative one day prior to the day for which maximum cleansing is desired.

In an embodiment of the invention drinking fluids comprises consuming an effective amount of rehydration fluid one day prior to the day for which maximum cleansing is desired. In certain embodiments the rehydration fluid is Gatorade®. In some embodiments the effective amount of rehydration fluid is about 3 to about 5L.

In some embodiments of the invention, the osmotic purgative comprises PICO-SALAX®. In some embodiments, the osmotic purgative comprises a small volume osmotic purgative which is Oral Sodium Phosphate, magnesium citrate, CITRO-MAG™, PICO-SALAX®, or a combination thereof. In some embodiments, the osmotic purgative is consumed in split doses. In some embodiments, the split doses are consumed about twelve hours apart. In some embodiments, the split doses are consumed about four to about six hours apart. In some embodiments, the split doses are consumed about five hours apart.

In some embodiments of the invention, the stimulant laxative comprises one or more of bisacodyl, senna, and sodium picosulphate. In some embodiments, the stimulant laxative comprises bisacodyl and the osmotic purgative comprises magnesium citrate. In some embodiments, the stimulant laxative comprises sodium picosulphate and the osmotic purgative comprises magnesium citrate. In certain embodiments, drinking fluids comprises consuming an effective amount of rehydration fluid one day prior to the day for which maximum cleansing is desired.

An aspect of the invention provides a kit for colon cleansing comprising a stimulant laxative; an osmotic purgative; and instructions for use of the kit comprising directions to consume an effective amount of the stimulant laxative 3 days and 2 days prior to the day for which maximum cleansing is desired, and to consume an effective amount of the osmotic purgative 1 day prior to the day for which maximum cleansing is desired.

Another aspect of the invention provides a kit for colon cleansing comprising a stimulant laxative; an osmotic purgative; and instructions for use of the kit comprising directions to consume an effective amount of the stimulant laxative at least 2 days prior to the day for which maximum cleansing is desired, and to consume an effective amount of the osmotic purgative 1 day prior to the day for which maximum cleansing is desired.

Yet another aspect of the invention provides a kit for colon cleansing comprising a stimulant laxative; an osmotic purgative; and instructions for use of the kit comprising directions to consume an effective amount of the stimulant laxative 4, 3, and 2 days prior to the day for which maximum cleansing is desired, and to consume an effective amount of the osmotic purgative 1 day prior to the day for which maximum cleansing is desired.

In some embodiments, the stimulant laxative is sodium picosulphate, senna, bisacodyl, or a combination thereof. In some embodiments, the osmotic purgative is Oral Sodium Phosphate, magnesium citrate, PICO-SALAX®, CITRO-MAG™, or a io combination thereof. In certain embodiments, the osmotic purgative is PICO-SALAX®. In some embodiments the kit further comprises rehydration fluid.

### BRIEF DESCRIPTION OF THE DRAWINGS

Embodiments of the invention will now be described, by way of example, with reference to the accompanying drawings.

FIG. 1A is a bar graph showing the percentage of specified colon sections in Groups 1A, 2, and 3 that were scored by the endoscopist either as “inadequate” or “poor” in the Ottawa Bowel Prep Scale.

FIG. 1B is a bar graph showing the percentage of specified colon sections in Groups 1B, 2, and 3 that were scored by the endoscopist either as “inadequate” or “poor” in the Ottawa Bowel Prep Scale.

FIG. 2A is a bar graph showing the percentage of patients in Groups 1A, 2, and 3 that stated in the Patient Colon Prep Tolerance questionnaire that their treatment regime was “very easy” or “easy” to take.

FIG. 2B is a bar graph showing the percentage of patients in Groups 1A, 2, and 3 that stated in the Patient Colon Prep Tolerance questionnaire that their treatment regime was “difficult” or “very difficult” to take.

FIG. 3A is a bar graph showing the percentage of patients in Groups 1A, 2, and 3 that stated in the Patient Colon Prep Tolerance questionnaire that the taste of their treatment regime was “good” or “excellent”.

FIG. 3B is a bar graph showing the percentage of patients in Groups 1A, 2, and 3 that stated in the Patient Colon Prep Tolerance questionnaire that they experienced nausea during or following their treatment regimen.

FIG. 4A is an example of instructions for patients in Group 1A.

FIG. 4B is an example of instructions for patients in Group 2.

FIG. 4C is an example of instructions for patients in Group 3.

FIG. 4D is an example of instructions for patients in Group 1B.

FIG. 4E is an example of instructions for patients in Group 1C.

FIG. 5 is an example of a Patient Colon Prep Tolerance questionnaire for completion by patients prior to their colonoscopy procedure.

FIG. 6 is an example of an Ottawa Bowel Prep Scale sheet for completion by an attending endoscopist following a colonoscopy procedure.

### DETAILED DESCRIPTION OF THE PREFERRED EMBODIMENTS

### Definitions

As used herein the term “colon” refers to a part of the large intestine extending from cecum to rectum.

As used herein the term “bowel” refers to intestines extending from stomach to anus.

As used herein the term “colonoscopy” refers to a procedure wherein an endoscope is moved through the regions of the colon and an endoscopist examines a visual image of the lining of the colon. The regions of the colon include: the right colon, which includes the cecum and the ascending colon; the mid colon, which is also known as the transverse colon; and the left colon, which is also known as the descending and recto-sigmoid colon.

As used herein the term “drinking fluids and restricting solid food” means limiting food intake to a clear fluid diet. A clear fluid diet may include, without limitation, rehydration fluid, water, clear fruit juices, clear broth, tea and coffee (without milk/dairy product), and JELL-O®.

As used herein the term “one day prior to the day for which maximum cleansing is desired” means the entire day prior to the procedure for which good visibility of the colon is required (e.g., colonoscopy), and it includes the part of the day of the procedure that is prior to the procedure.

As used herein the term “stimulant laxative” means a composition that when consumed stimulates colon muscles causing the colon to expel fecal matter contained therein. Examples of stimulant laxatives include bisacodyl, senna, and picosulphate salts such as sodium picosulphate.

As used herein the term “BISACODYL198 ” means a product that includes bisacodyl, a synthetic pyridinylmethylene-diacetate ester derivative stimulant laxative, as well as non-medicinal ingredients. Certain DULCOLAX™ products include bisacodyl.

As used herein the term “osmotic purgative” means a composition that draws water into the colon causing the colon to expel watery feces, mucous, and substantially all matter contained in the colon. Small volume osmotic purgatives use salts to draw water into the colon. Such salts include sodium phosphate and magnesium citrate. Oral Sodium Phosphate is available from C.B. FLEET® Co, Lynchburg, Va., USA as PHOSPHO-SODA® and from Pharmascience, Inc. Montreal, Quebec, Canada as PHOSPHATE SOLUTION™.

As used herein the osmotic purgative “magnesium citrate” encompasses the result of mixing magnesium oxide and citric acid in aqueous solution. Magnesium citrate is the active purgative ingredient in CITRO-MAG™ and PICO-SALAX®. CITRO-MAG™ is available from Rougier Pharma, Mississauga, Ontario, Canada and it is an anhydrous magnesium citrate oral solution.

As used herein the term “PICO-SALAX®” refers to a product that includes both osmotic purgative and stimulant laxative and that is available from Ferring Inc., North York, Ontario, Canada. This product has as its stimulant laxative sodium picosulphate (10 mg), which is suggested to work by decreasing water and electrolyte absorption and increasing motility by inducing peristaltic contractions in the colon. It has as its osmotic purgative agent magnesium citrate which is formed in solution from magnesium oxide and citric acid (e.g., per instructions, magnesium oxide 3.5 g and citric acid 12 g are mixed with approximately 150 mL of clear liquid). Other products are available around the world that are similar to PICO-SALAX® of Ferring Inc. (Canada) because they also include sodium picosulphate and magnesium oxide and citric acid in approximately the same ratios. Such compounds may include Picolax (Australia), Picolax (Italy), Picolite (France), Picolon (Denmark), PicoPrep (New Zealand), and PicoPrep (Australia). Product ingredients should be checked to verify that at least the osmotic purgative magnesium citrate (formed in solution from magnesium oxide and citric acid) is present before substituting for PICO-SALAX® (Canada), having been selected herein for that purpose.

As used herein the term “Group 1A” refers to a group of 100 patients whose pre-colonoscopy colon cleansing regimen was treatment with PICO-SALAX® plus two doses of BISACODYL™. Specifically, these patients consumed 10 mg bisacodyl orally at 5 pm three days before colonoscopy, 10 mg bisacodyl orally at 5 pm two days before colonoscopy, 1 sachet of PICO-SALAX® at 5 pm one day prior to colonoscopy, and a second sachet of PICO-SALAX® at 10 pm one day prior to colonoscopy.

As used herein the term “Group 1B” refers to a group of 12 patients that were treated with PICO-SALAX® plus one dose of BISACODYL™. Specifically, these patients consumed 10 mg bisacodyl orally at 5 pm two days prior to colonoscopy, 1 sachet of PICO-SALAX® at 5 pm one day prior to colonoscopy, and a second sachet of PICO-SALAX® at 10 pm one day prior to colonoscopy.

As used herein the term “Group 1C” refers to a group of 7 patients that were treated with CITRO-MAG™ plus two doses of BISACODYL™. Specifically, these patients consumed 10 mg bisacodyl orally at 5 pm three days before colonoscopy, and 10 mg bisacodyl orally at 5 pm two days before colonoscopy, If the patient's colonoscopy was scheduled to begin substantially earlier than 11 am, then he/she was directed to consume 1 portion of CITRO-MAG™ at 5 pm one day prior to colonoscopy, and a second portion of CITRO-MAG™ at 10 pm one day prior to colonoscopy. If the patient's colonoscopy was scheduled to begin at approximately 11 am or later, then he/she was directed to consume 1 sachet of CITRO-MAG™ at 7 pm one day prior to colonoscopy, and a second sachet of CITRO-MAG™ at 6 am on the day of the colonoscopy.

As used herein the term “Group 2” refers to a group of 104 patients that were treated with PICO-SALAX® alone. Specifically, these patients consumed 1 sachet of PICO-SALAX® at 5 pm one day prior to colonoscopy, and a second sachet of PICO-SALAX® at 10 pm one day prior to colonoscopy.

As used herein the term “Group 3” refers to a group of 96 patients that were treated with Oral Sodium Phosphate. Specifically, these patients consumed a first bottle (specifically, PHOSPHATE SOLUTION ™ of Pharmascience, Inc.) of Oral Sodium Phosphate at 5 pm one day prior to colonoscopy, and a second bottle of Oral Sodium Phosphate at 10 pm one day prior to colonoscopy. Each bottle contained monobasic sodium phosphate monohydrate (2.4 g), dibasic sodium phosphate heptahydrate (0.9 g) and non medicinal ingredients including glycerine, flavour, purified water, sodium benzoate, and sodium saccharin as a 45 mL concentrated solution.

### DESCRIPTION

An embodiment of the invention described herein relates to a method of preparing a patient's colon for a colonoscopy. However, a person of ordinary skill in the art of the invention shall appreciate that a clean colon is desirable for any event wherein a visual inspection of the colonic membranes is performed, e.g., colonoscopy, colon surgery, CT (computed tomography) colonography, etc. Colon cleansing techniques described herein are appropriate for a variety of procedures. As discussed previously, optimal colon cleansing regimens for colonoscopy should provide excellent cleansing, be well tolerated by patients, and have a high safety profile (Hookey et al., 2002; Rex, 2006).

A recent increase in the number of colonoscopy screening programs has intensified the search for optimum pre-colonoscopy colon cleansing regimens to simplify colonoscopy procedures by avoiding poor colon cleanliness. One out of five incomplete colonoscopies cite poor colon preparation as the reason completion was prevented (Belsey, 2007). Efficient colon cleansing prior to colonoscopy is important to allow the endoscopist to see the lining of the colon well enough to detect abnormalities and therefore to optimize polyp detection rates (Chiu et al., 2006; Froehlich et al., 2005; Rex, 2006). Colon cleansing typically involves a change in diet and use of laxatives. Colons with remaining debris in the colonic mucosa such as fecal matter, mucous, etc. at the time of colonoscopy require washing of the membranes to allow a clear view of the lining. Such washing is typically performed by producing a stream of water. Suctioning of debris and/or of the washings may also be necessary. In some situations, a gas such as air or carbon dioxide is then allowed to flow from the endoscope. The gas expands the colon for better viewing. Such washing, suctioning, and inflating prolongs the length of time of the procedure. Poor colon cleanliness can lead to the requirement that the patient go through the procedure, including the pre-colonoscopy colonic cleansing, again. Poor colon cleanliness can also lead to a decision to decrease the time interval to subsequent colonoscopy screening. For these reasons, poor colon cleanliness adds pressure on colonoscopy facilities and staff by adding to the number of procedures and the length of time of procedures. The quality of colon preparation impacts the cost of colonoscopies; imperfect colon preparation is estimated to increase the cost of colonoscopy screening by 12-22% (Rex et al. 2002).

Tolerance of a pre-colonoscopy colonic cleansing regimen by a patient is an important factor to achieving optimal cleansing. Intolerance impacts patient compliance and thus affects success of colonoscopy screening programs. Large volume preparations are poorly tolerated and certain studies indicate that they provide poorer colon cleanliness (Hsu et al., 1998; Belsey et al., 2007; Tan et al. 2006). Small volume preparations have emerged as a favourable alternative to large volume purgatives because they have better patient tolerance. Oral Sodium to Phosphate (also known as NaP), is a small volume osmotically active solution of sodium phosphate salts that has emerged as a favorable alternative to large volume purgatives. An example of a large volume purgative is an osmotically balanced polyethylene glycol (also known as PEG) electrolyte solution, which is also known as Colyte®. Oral Sodium Phosphate is better tolerated by most patients than large volume purgatives and provides superior cleansing to PEG solutions. However, although Oral Sodium Phosphate is safe in most patients, it causes transient hyperphosphatemia; some reports suggest that this disturbance, possibly coupled with dehydration, leads to nephrocalcinosis and chronic renal failure (Markowitz et al., 2005; Markowitz et al., 2004; Desmeules et al., 2003). In contrast, patient tolerance and safety for PICO-SALAX® is excellent.

Studies described herein investigated pre-colonoscopy cleansing regimens and quantified the results. Studies were conducted to compare pre-colonoscopy colon cleansing regimens. Goals of these studies included evaluating efficacy, patient tolerance, and patient safety of these regimens. In total, three hundred and fifty-one patients were enrolled into main Groups 1A, 2, and 3, and twelve were enrolled into Group 1B. Endoscopists were trained in a validated scoring scale. Following colonoscopy of each patient the endoscopist recorded a score for each colon section, and an overall score. These scores were then used to quantify the level of cleanliness achieved in each section of the colon of each patient population. Patient tolerance and safety were evaluated for each of the three main Groups. According to these studies, an improved colon cleansing regimen for colonoscopy is described which has high efficacy, particularly in the ascending colon, very high patient approval, and an excellent safety profile. This improved regimen involves use of an adjuvant stimulant laxative together with use of a product including an osmotic purgative. In an embodiment of the invention, the stimulant laxative is consumed at least two days prior to colonoscopy and the osmotic purgative is consumed one day prior to colonoscopy together with a liquid diet. Although not wishing to be bound by theory, the investigators of studies described herein suggest that initial cleansing of solid stool, caused by the stimulant laxative, before the patient initiates the clear liquid diet and osmotic purgative cleansing, is more effective at cleansing the colon than cleansing with small volume osmotic purgative alone. To maintain high patient tolerance, separate stimulant is laxatives were not consumed on the same day as the product that included osmotic purgative.

An aspect of the invention provides an appropriately timed colonic cleansing method involving consumption of one or more stimulant laxatives (e.g., bisacodyl, senna, sodium picosulphate), followed by a clear fluid diet and consumption of one or more products that include osmotic purgative (e.g., PICO-SALAX®, CITRO-MAG™, Oral Sodium Phosphate). In an embodiment of the invention, stimulant laxative is consumed by the patient three days and two days prior to colonoscopy, then osmotic purgative is consumed one day prior to colonoscopy while restricting solid food. In another embodiment of the invention, stimulant laxative is consumed by the patient two days prior to colonoscopy, then an osmotic purgative is consumed by the patient one day prior to colonoscopy while restricting solid food. In yet another embodiment of the invention, stimulant laxative is consumed by the patient four, three, and two days prior to the colonoscopy, then osmotic purgative is consumed one day prior to colonoscopy while restricting solid food. In some embodiments when the time of the colonoscopy was substantially earlier than 11 am, the time period separating consumption of each of the osmotic purgative's split doses was about 5 hours (e.g., 5 pm and 10 pm). In certain embodiments when the time of the colonoscopy was about 11 am, the time period separating consumption of each of the osmotic purgative's split doses was about 11 hours (e.g., 7 pm one day before the colonoscopy and 6 am on the morning of the colonoscopy).

Patient tolerance for PICO-SALAX® when taken alone is excellent. As shown herein, patient tolerance is unaffected by the addition of a separate stimulant laxative when it is consumed on days other than the day of consumption of PICO-SALAX®.

Consumption of both a separate stimulant laxative and PICO-SALAX® on a given day is avoided to maintain good patient tolerance. This aspect wherein administration of separate stimulant laxative and small volume osmotic purgative cleansing agent are spread over several days and are not both taken on any day, provides superior cleansing when compared to cleansing with small volume osmotic purgative alone. In contrast, other studies which combine stimulant laxatives and small volume osmotic purgatives on the same day failed to show any advantage (Hookey et al., 2004; Afridi et al., 1995).

In patient tolerance studies described herein, treatment groups received an osmotic purgative on the evening before colonoscopy in split doses. Comparisons were made between the results of these osmotic purgatives. Also, a comparison was made between patients treated with osmotic purgative alone, and patients treated initially with stimulant laxative followed by osmotic purgative. Table III provides a summary of data gathered from diaries that were kept by 100 patients as they progressed through a colon cleansing regimen. Each patient recorded the time of the first and last bowel movement following consumption of a laxative, as well as the total number of bowel movements associated with consumption of a particular laxative.

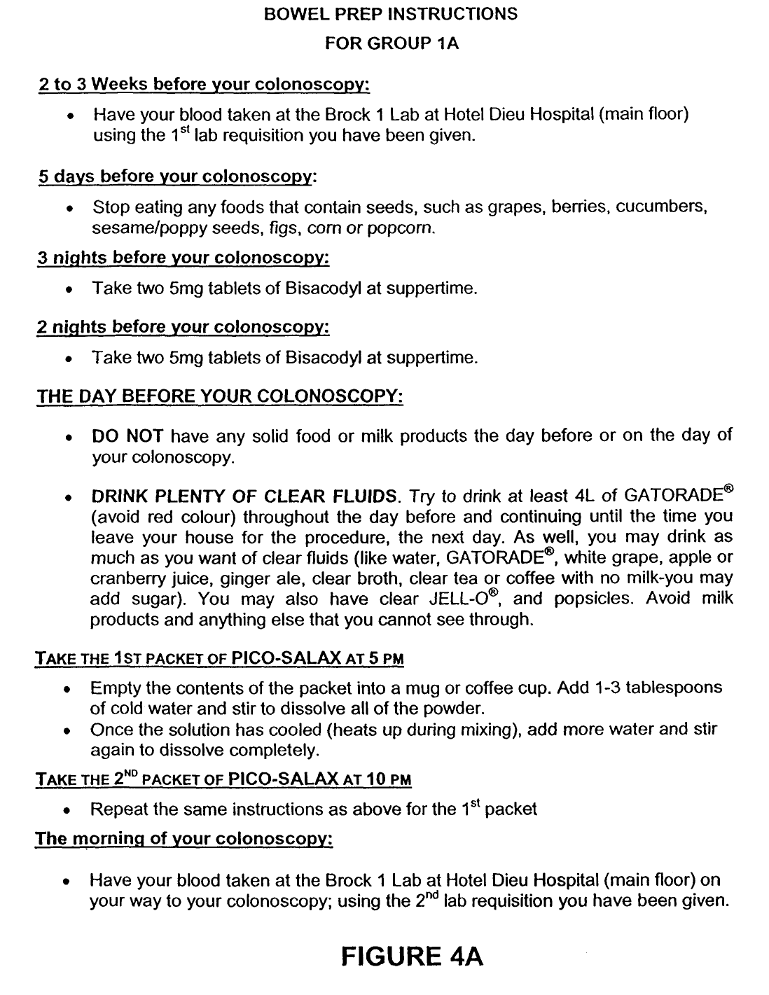

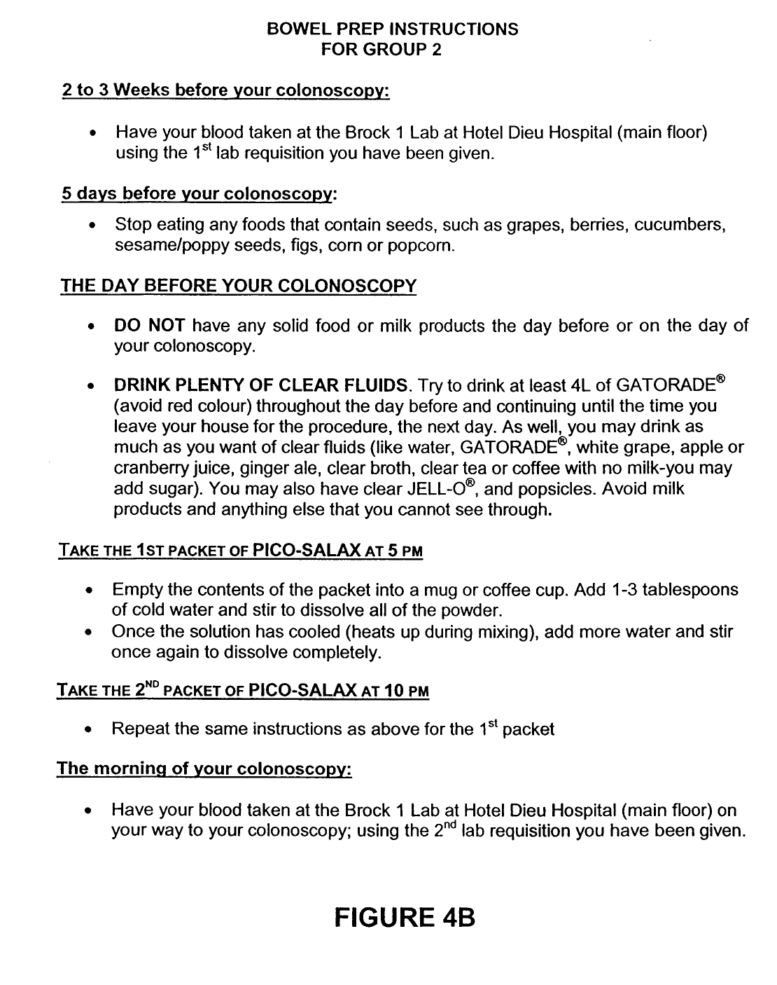

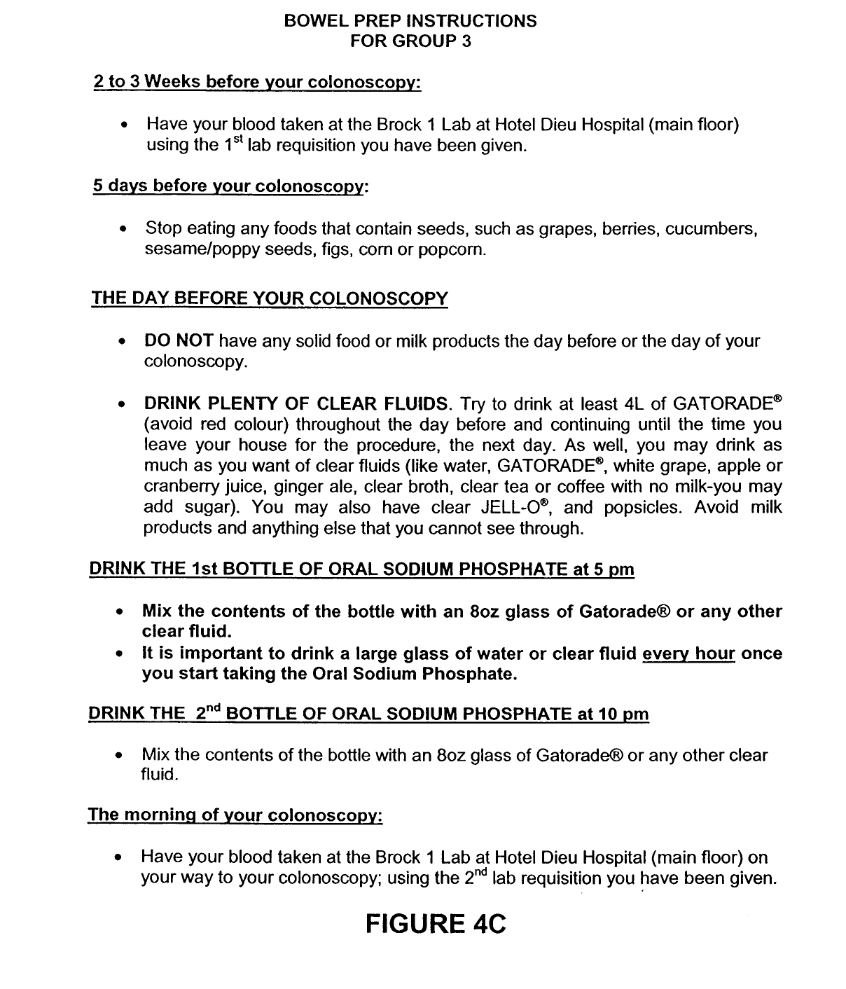

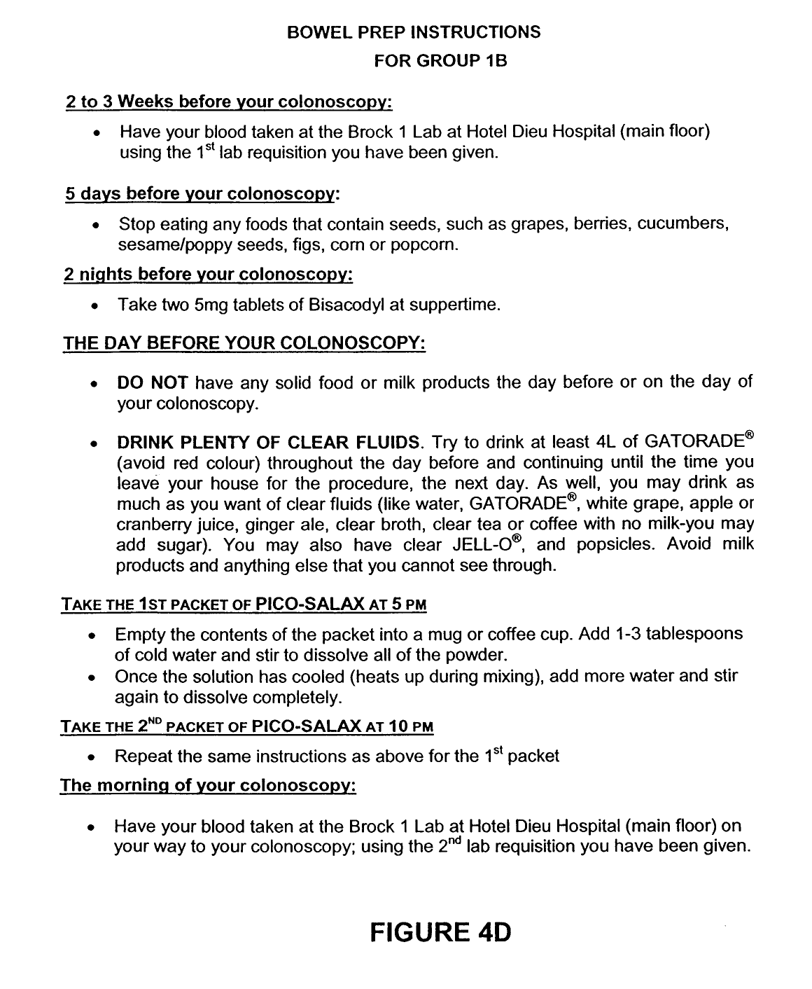

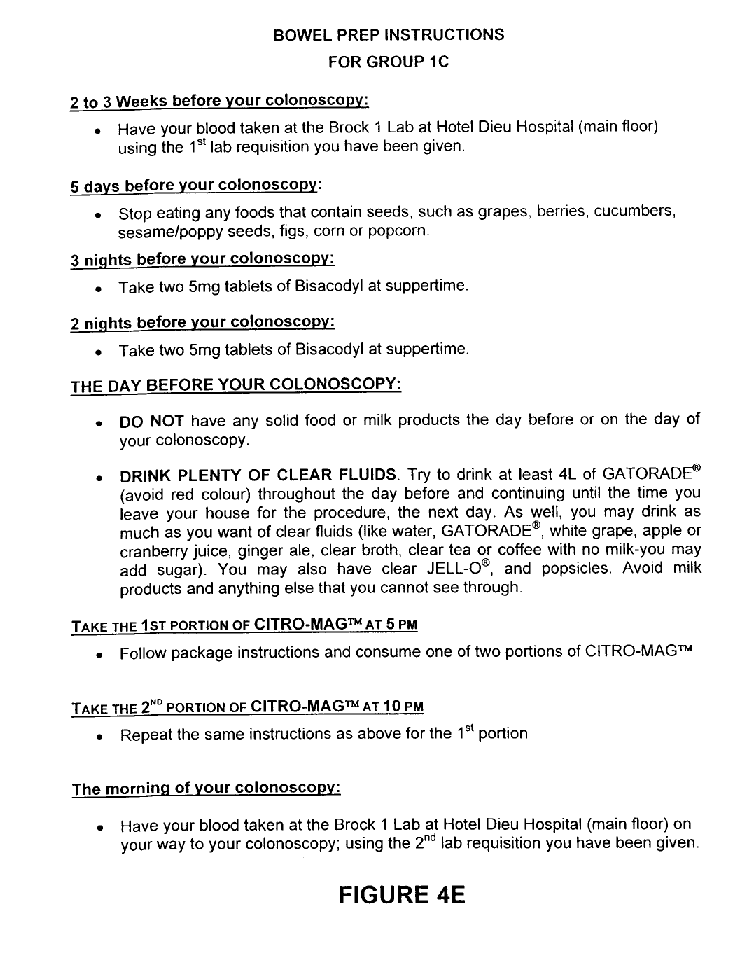

In addition, to provide insight into the diary data, the following example was created from the data of Table III by choosing the highest number of bowel movements with the least response time. This artificial example is intended to provide an idea of a possible patient experience during the colon cleansing regimen described in FIG. 4A with the modification that the second portion of the split dose of PICOSALAX® is consumed on the morning of the colonoscopy. A patient has a colonoscopy booked for Friday morning at 11:30 am. On the Sunday prior to the colonoscopy, the patient stops eating any food that contains seeds and corn, including popcorn. On Tuesday evening, the patient takes bisacodyl (10 mg) with an evening meal around 6 pm. At 10 pm, the patient has a first bowel movement (BM) since taking the bisacodyl. The patient has 5 separate episodes of BMs in total with the last one occurring at 2 pm on Wednesday. On Wednesday with the evening meal at about 6 pm, the patient takes bisacodyl (10 mg) for a second time. At 10 pm, the patient has a first BM since taking the second dose of bisacodyl. The patient has 6 separate episodes of BMs in total with the last one occurring at 3 pm on Thursday.

The patient refrains from eating any solid food or milk products on Thursday and on the part of Friday that is prior to the colonoscopy. To stay well hydrated, a clear fluid diet is maintained during the time period of no solid food intake. A clear fluid diet may include rehydration fluid, water, clear fruit juices, clear broth, tea and coffee (without milk product), JELL-O®, etc. On Thursday at about 7 pm, the patient consumes the first of two split doses of PICOSALAX®. Within half an hour, the patient has a first BM since taking the PICOSALAX®. The patient has 8 episodes of BMs in total with the last one at about 3 am on Friday. At 6 am on Friday, the patient consumes the 2nd of the split doses of PICOSALAX®. Within minutes, the patient has a first BM. 7 more BMs follow with the last one happening at 10 am on Friday.

The colonoscopy proceeds at about 11:30 am on Friday. The endoscopist reports that the visibility of the colon walls is good and the patient does not need to return for another colon screening since a thorough inspection of the mucosal lining is possible. The total number of separate episodes of bowel movements experienced by the patient during the colon cleansing regimen was 19.

Studies described herein showed that an adjuvant stimulant laxative, administered prior to and not on the same day as product including an osmotic purgative, significantly enhanced colon cleansing efficacy in the right colon, without compromising or even affecting patient tolerability or safety. Of particular note, stimulant laxative preceding osmotic purgative provided superior colon cleansing in the right colon when compared to use of osmotic purgative alone. to On the day prior to the colonoscopy procedure, patients were required to refrain from eating solid food and were encouraged to stay well hydrated. To do so, it is recommended that patients drink between about 3 to about 5 L of rehydration fluid (in a colour other than red) during the day prior to the colonoscopy. An example of rehydration fluid is Gatorade® which is available from Quaker Oats Co. of Canada in Peterborough, Ontario, Canada. Gatorade® has been proven to mitigate against intravascular volume depletion caused by small volume osmotic agents (Barclay et al., 2002). Gatorade® is an over-the-counter sports drink that contains simple carbohydrates (sucrose, glucose, fructose) and small concentrations of sodium and potassium. Gatorade® was recommended to patients because it is an effective oral rehydration mixture (Vanner et al. U.S. Pat. No. 7,332,184).

BISACODYL™ (available from Boehringer Ingelheim, Burlington, Ontario, Canada) is a product that is recommended for facilitation of defecation. It is a synthetic pyridinylmethylene-diacetate ester derivative stimulant laxative. Without wishing to be bound by theory, it is believed to act with parasympathetic effect directly on mucosal sensory nerves, increasing peristaltic contractions in the colon. It was provided to patients in the appropriate Groups as yellow, enteric-coated tablets. Each tablet contained bisacodyl (5 mg) and non-medicinal ingredients which may include acacia, acetylated monoglyceride, beeswax, carnauba wax, cellulose acetate phthalate, cornstarch, dibutyl phthalate, docusate sodium, gelatin, glycerin, iron oxides, kaolin, lactose, magnesium stearate, methylparaben, polyethylene glycol, povidone, propylparaben, sodium benzoate, sorbitan monooleate, sucrose, talc, titanium dioxide, and yellow dye.

Each sachet of PICO-SALAX® (available from Ferring Inc., North York, Ontario, Canada) contained 16.1 g of powder which had 3 active ingredients: sodium picosulfate (10 mg), magnesium oxide (3.5 g), and citric acid (12 g). The magnesium oxide and citric acid form magnesium citrate in solution. Each sachet also contained non-medicinal ingredients including orange flavour, potassium bicarbonate and sodium saccharin. Without wishing to be bound by theory, the inventors suggest that PICO-SALAX® acts by two mechanisms. First, sodium picosulphate increases motility in the colon by decreasing water and electrolyte absorption and stimulating peristaltic contractions in the colon. Second, magnesium citrate (magnesium oxide and citric acid) increases the amount of water in the bowel by acting as an osmotic agent.

Small volume osmotic purgatives, which are recommended to be consumed in split doses, frequently cause patients to experience sudden intense desires to defecate. Patient tolerance for purgatives is influenced by convenient access to toilet facilities following consumption of each split dose.

In the studies described herein, the split doses of small volume osmotic purgatives were consumed during the late afternoon and evening of the day prior to the colonoscopy with a time interval between the doses of about 5 hours. Since most patients were scheduled for colonoscopies in the morning (e.g., before about 11 am), it was effective to have both doses of osmotic purgative taken the night before colonoscopy. Moreover, by consuming both doses on the night before, patients were better able to travel in the morning.

Alternatively, provided the patient has access to toilet facilities following consumption of each dose, the first dose can be consumed the night before colonoscopy and the second dose can be consumed in the early morning of the day of the colonoscopy. For colonoscopies performed later in the day (e.g., after about 11 am), consumption of the second dose on the day of the colonoscopy (e.g., in the early morning) is expected to be most effective.

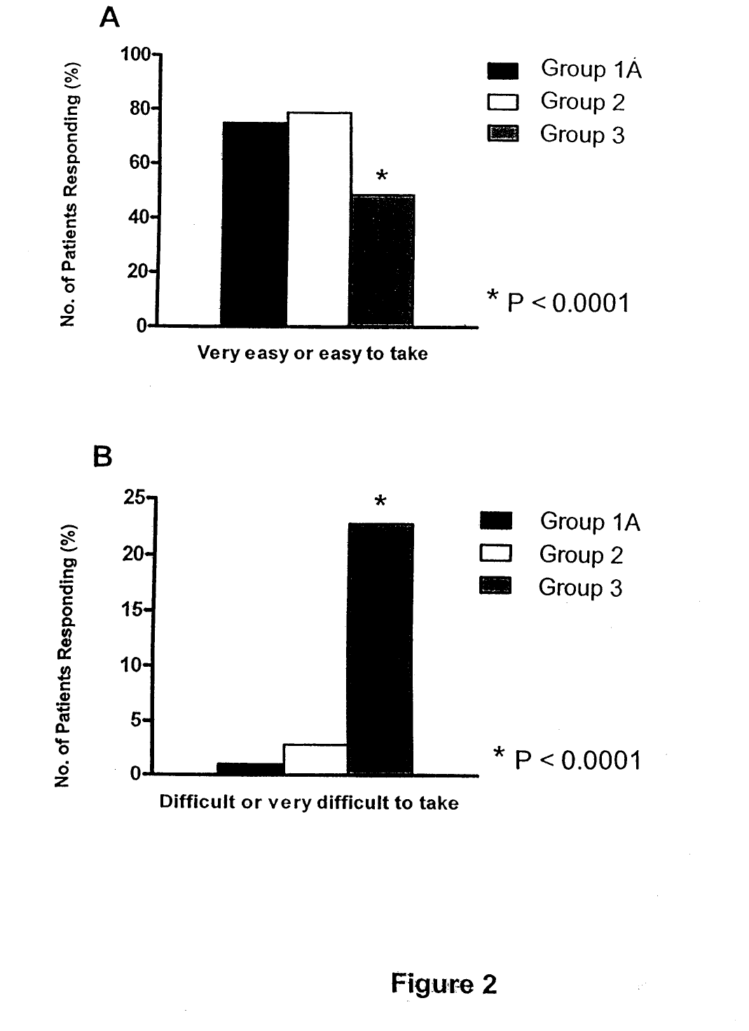

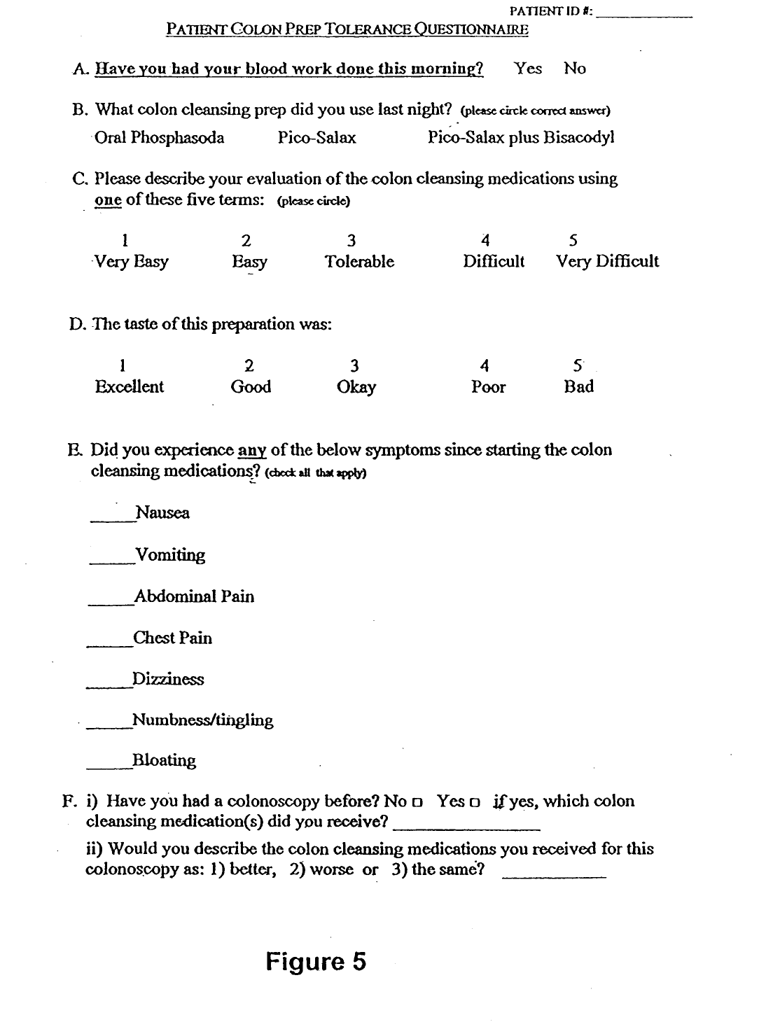

Patient tolerance was quantified by having the patients complete a questionnaire upon arrival for the colonoscopy; an example of the questionnaire is shown in FIG. 5. This commonly applied tool uses a five point Likert scale to assess overall ease of taking the preparation. It also assesses taste and the following specific symptoms: nausea, abdominal or chest pain, vomiting, and bloating. Details of patient acceptance/tolerability are provided in Example 3 and FIGS. 2A-B and 3A-B.

Patient safety was monitored by measuring hemodynamic and biochemical data before and after pre-colonoscopy colon cleansing. Specifically, hemodynamic and biochemical data were measured at baseline, in the clinic prior to booking a colonoscopy appointment, and on arrival in the endoscopy suite on the colonoscopy day. Biochemical measures compared changes in serum electrolytes, intravascular volume, renal function and specific variables (calcium, magnesium, phosphate) which may be altered due to the composition of the different regimens. Renal function was investigated via levels of hematocrit, BUN Cr (Blood Urea Nitrogen Creatinine), and estimated GFR (Glomecular Filtration Rate).

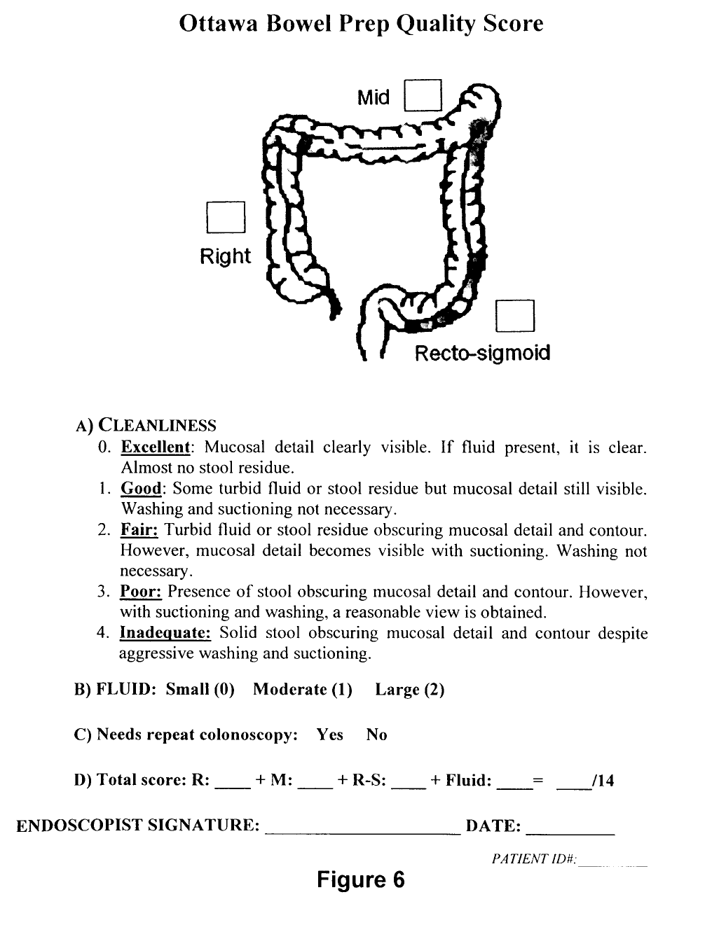

During colonoscopy procedures, colon cleanliness was evaluated using a validated research tool which has been proven capable of detecting a difference in colon cleanliness, the Ottawa Bowel Prep Scale (“OBPS”) scoring system (Rostom et al., 2004; Rostom et al., 2006; Gupta et al., 2007) (see Example 4 and FIG. 6). OBPS was selected because it provides both global and local measures of efficacy. Thus, it provides scores that quantify cleanliness of the whole colon and specific regions of the colon.

All endoscopists that provided colonoscopy scores for the studies described herein were trained in using OBPS using photographs illustrating examples of cleanliness ratings on the OBPS. Endoscopist training included having endoscopists rate colonoscopy photos from 20 colonoscopies both before and after their training session, to verify that the training was effective in reducing scoring variance.

Furthermore, a poster with exemplary endoscopic photos from each rating on the OBPS was made available to the endoscopist in each endoscopy room. The OBPS scale uses ratings from 0-4 (briefly, 0=excellent, 4=inadequate, see FIG. 6 for details and Example 4 for further description) for each section of the colon, along with a score for overall fluid (0=small, 1=moderate, 2=large). The OBPS scale provides a global score as to the cleanliness of each patient's colon out of a possible 14. The global score is obtained by summing the score from the left, mid and right colon and the overall fluid. Low global scores indicate excellent colon cleansing while high global scores indicate inadequate colon cleansing, e.g., 0 (excellent preparation, no fluid) to 14 (inadequate in all segments with a large amount of fluid).

It is noted that on the OBPS, instances of washing, no matter how minimal, are scored as “poor” or “inadequate”. Instances of suctioning and not washing, are scored as “fair”.

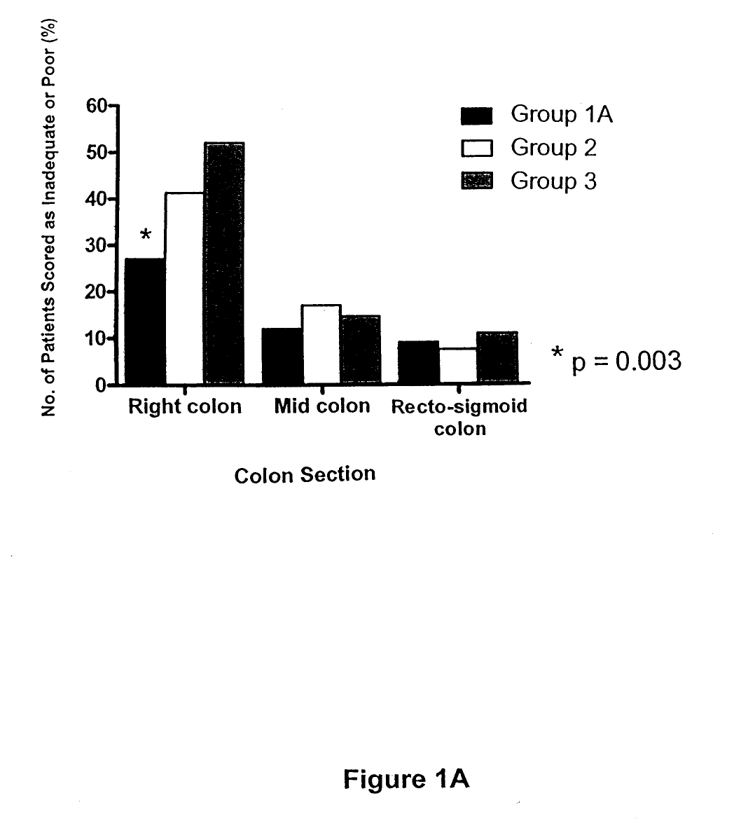

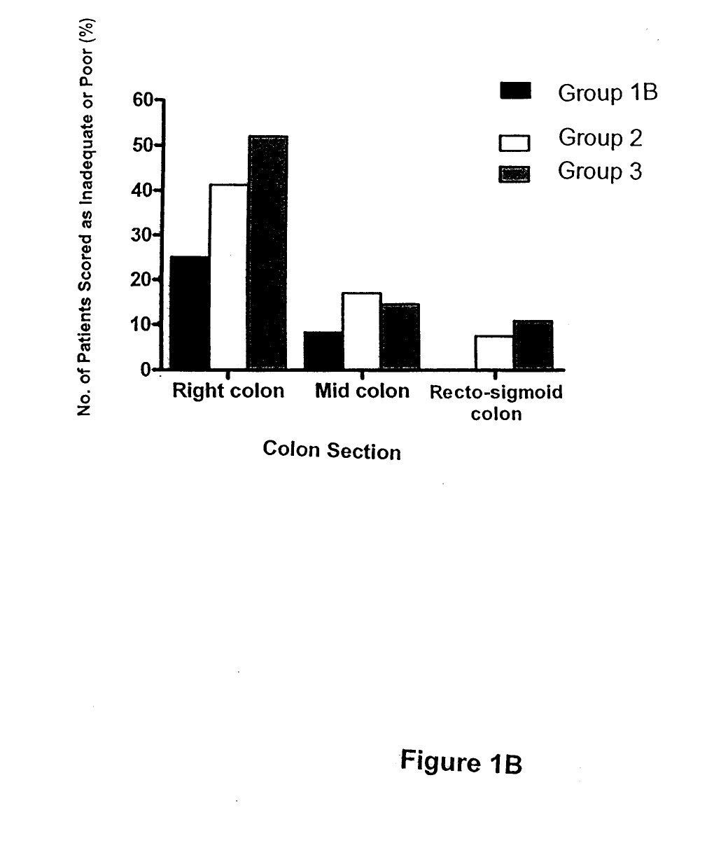

Efficacy results of the patient studies described herein indicate that when individual components of the OBPS (i.e. right colon, middle colon, left colon) were analyzed, colon cleanliness of patients in Group 1A was significantly better in the right colon (see FIG. 1A). Specifically, approximately 50% fewer scores of “inadequate” or “poor” were obtained for the right colon for Group 1A patients when compared to Group 3 patients, and approximately 30% fewer scores of “inadequate” or “poor” were obtained for the right colon when compared to Group 2 patients.

Similar results were obtained for the right colon of patients in Group 1B when compared to Groups 2 and 3 (see FIG. 1B). It is believed that these differences between Groups would be even greater if not for the above-described deficiency in the OBPS that any washing, no matter how minimal, required that the colon section be scored as poor.

Group 1C's result was consistent with the result for Group 1A. Group 1C's mean OBPS score was 4.9 and visibility of the right colon, middle colon, and left colon segments were determined to be good or fair.

No significant differences were seen in the mid and recto-sigmoid colon between the Groups and there was no difference between Groups 1A, 2, and 3 in mean global OBPS score. Mean totals±standard deviation were as follows: 5.0±2.4 (Group 1A); 5.1±2.8 (Group 2); and 5.1±2.6 (Group 3); where p=0.96. A to description of statistical analysis performed for Groups 1A, 2 and 3 appears in Example 5.

Interestingly, women had a better mean global OBPS score than men when considering Groups 1A, 2, and 3 (5.1 vs. 5.9, p=0.009). This difference was consistent among these Groups. Scores of colon cleanliness, as measured by OBPS, were significantly worse in colonoscopies performed after 11 am compared to those done before 11 am (mean score 5.5 vs. 4.7, p<0.01). This difference was independent of pre-colonoscopy colon cleansing regime.

Results of these studies showed that treatment with PICO-SALAX® plus BISACODYL™ provided superior colon cleansing in the right colon. As discussed previously, the ascending (or right) colon is most difficult to cleanse, so the quality of the colon preparation is often the poorest there (Gupta et al., 2007; Rostom et al., 2006; Park et al., 2007; Ell et al., 2003). Also as discussed previously, the ascending colon is frequently the location of flat lesions, which are among the most difficult type of abnormality to visualize (Park et al., 2008; Rex, 2006; Brooker et al., 2002; Saitoh et al., 2001). Accordingly, the ascending colon is important to have well cleansed to allow visualization of flat lesions and is most difficult to have well cleansed. For these reasons, the efficacy results of the regimen of osmotic purgative preceded by stimulant laxative (Groups 1A and 1B), the combination which provided the best cleansing in the right colon, are truly significant.

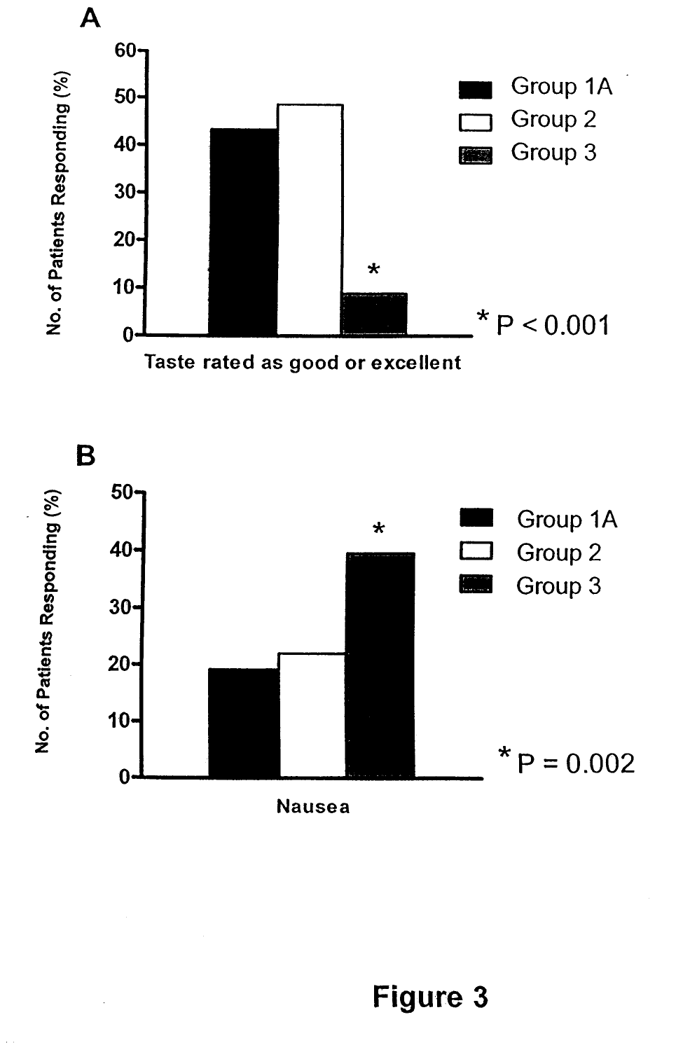

Results of patient tolerance questionnaires are presented graphically in FIGS. 2A, 2B, 3A, and 3B. No differences were seen between Groups in reported severity of abdominal or chest pain, vomiting, or bloating. When the two osmotic purgatives were compared for patient tolerance, PICO-SALAX® was tolerated significantly better than Oral Sodium Phosphate. Notably, this better tolerance was not affected when BISACODYL™ was added to the PICO-SALAX® regimen. FIGS. 2A, 2B, 3A, and 3B support the finding that patient tolerance was to substantially equivalent for Group 1A and Group 2; while patients are least tolerant of the Oral Sodium Phosphate regimen. Therefore, the regimen of PICO-SALAX® plus BISACODYL™ (Groups 1A and 1B), the combination which provided the best cleansing in the right colon, is very well tolerated.

Patient safety was unaffected by the addition of stimulant laxative to a regimen of osmotic purgative. Based on hemodynamic or biochemical measurements, there was no evidence of intravascular volume depletion in any of the Groups based on changes in postural blood pressure, pulse or serum urea, creatinine and estimated GFR (see Table II). Groups 1A and 2 were associated with a clinically insignificant increase in hematocrit when compared to Group 3 (0.01±0.02 vs. 0.00 ±0.02, p=0.001). There were statistically significant differences among the Groups in changes in serum calcium, sodium, chloride, potassium, magnesium, and phosphate (see Table II). A rise in serum phosphate and reciprocal decrease in calcium was observed in a significant number of Group 3 patients. These changes were not seen in Groups 1A and 2. There was a slight rise in serum magnesium in about a third of patients receiving PICO-SALAX®, but these changes were clinically insignificant (maximum value 1.21 mmol/L; normal range=0.8-1.0 mmol/L). There were significantly more patients with hypokalemia in Group 3 (73%) compared to Group 1A (10%) and the magnitude of the decrease was greater in Group 3 (lowest level=2.7 mmol/L; normal range=3.5-5.2 mmol/L).

Several minor biochemical changes were detected during these studies, specific to each of the colon preparations. Patients receiving Oral Sodium Phosphate had an elevated phosphate level on the morning of the colonoscopy. No change in the phosphate level was observed in patients receiving PICO-SALAX® either alone (Group 2) or with BISACODYL™ (Group 1A). A significant increase in mean magnesium levels compared to baseline was found on the morning of colonoscopy for patients who took PICO-SALAX®, but they represented clinically insignificant deviations. A small but significant decrease in mean serum sodium levels in the Groups receiving PICO-SALAX® was detected, but these also were clinically insignificant. Together, these safety data support the paucity of reported adverse events with PICO-SALAX®, suggesting it has an excellent safety profile. Notably, the excellent safety profile of PICO-SALAX® was not affected by the addition of pre-treatment with stimulant laxative.

Referring to FIG. 1A, results of studies described herein are displayed as a bar graph that shows the percentage of specified colon sections in Groups 1A, 2, and 3 that were scored by the endoscopist either as “inadequate” or “poor” in the Ottawa Bowel Prep Scale. As seen in this figure, compared to both Groups 2 and 3, Group 1A resulted in significantly fewer scores for the right colon section of either “inadequate” or “poor”.

Referring to FIG. 1B, results of studies described herein are displayed as a bar graph that shows the percentage of specified colon sections in Groups 1B, 2, and 3 that were scored by the endoscopist either as “inadequate” or “poor” in the Ottawa Bowel Prep Scale. As seen in this figure, compared to both Groups 2 and 3, Group 1B resulted in fewer scores for the right colon section of either “inadequate” or “poor”.

Referring to FIG. 2A, a bar graph is presented that shows the percentage of patients of Groups 1A, 2, and 3 that stated in the Patient Colon Prep Tolerance questionnaire that their treatment regime was “very easy” or “easy” to take. A significantly higher proportion of patients from Group 2 (78.9%) and Group 1A (75%) indicated that their regimen was very easy or easy to take, compared to Group 3 (47.5%). Notably, the number of patients in Groups 1A and 2 that reported that their treatment regime was “very easy” or “easy” to take is almost equal. This result indicates that PICO-SALAX™'s excellent patient tolerance is unaffected by an added two days of treatment with stimulant laxative.

Referring to FIG. 2B, a bar graph is presented that shows the percentage of patients of Groups 1A, 2, and 3 that stated in the Patient Colon Prep Tolerance questionnaire that their treatment regime was either “difficult” or “very difficult”. These results indicate that the regimens of Groups 1A and 2 were easier to tolerate than that of Group 3. Notably, the number of patients in Groups 1A and 2 that reported that their treatment regime was “difficult” or “very difficult” to take was almost equal. As was discussed for FIG. 2A, this result indicates that PICO-SALAX®'s excellent patient tolerance is unaffected by an added two days of treatment with stimulant laxative.

Referring to FIG. 3A, a bar graph is presented that shows the percentage of patients that stated in the Patient Colon Prep Tolerance questionnaire that the taste of their treatment regime was either “good” or “excellent”. A significantly higher proportion of patients in Group 2 (48.6%) and Group 1A (43.3%) rated the taste as excellent or good compared to only 8.9% of those in Group 3 (p<0.001).

Referring to FIG. 3B, a bar graph is presented that shows the percentage of patients that stated in the Patient Colon Prep Tolerance questionnaire that they experienced nausea during their treatment regime. A higher proportion (39.6%) of patients in Group 3 reported nausea, when compared to Group 2 (22%) and Group 1A (19.2%, p=0.002). Again, this result supports the finding that the excellent patient tolerance seen for PICO-SALAX® is unaffected by the addition of treatment with stimulant laxative.

Referring to FIG. 4A, an example is shown of instructions for patients in Group 1A.

Referring to FIG. 4B, an example is shown of instructions for patients in Group 2.

Referring to FIG. 4C, an example is shown of instructions for patients in

Group 3.

Referring to FIG. 4D, an example is shown of instructions for patients in Group 1B.

Referring to FIG. 4E, an example is shown of instructions for patients in Group 1C.

Referring to FIG. 5, an example is shown of a Patient Colon Prep Tolerance questionnaire completed by patients in all Groups prior to their colonoscopy procedure.

Referring to FIG. 6, an example is shown of an Ottawa Bowel Prep Scale sheet completed by an attending endoscopist following a colonoscopy procedure.

The present invention may be supplied as a kit, containing stimulant laxative and osmotic purgative. The stimulant laxative may be a pill, powder, capsule, or liquid. The osmotic purgative may be supplied as a solution or in dry form, to which clear liquid, e.g., water, may be added to form a solution prior to oral administration. The kit may include two or more containers, packs, or dispensers together with instructions for preparation and use. Preferably, the kit contains two split doses of osmotic purgative, preferably each in a separate container, and stimulant laxative in one or more additional containers. If stimulant laxative is for administration 3 and 2 days or 4, 3 and 2 days before a procedure requiring a cleansed colon, each dose may be conveniently provided in a separate container. If stimulant laxative is for administration 2 days before a procedure requiring a cleansed colon, it may be conveniently provided in a single container.

The compositions included in the kit may be supplied in containers of any sort such that the integrity of the different components are preserved and they are not adsorbed or altered by the materials of the container or by other components. For example, suitable containers include simple bottles that may be fabricated from glass, organic polymers such as polycarbonate, polystyrene, etc., ceramic, metal or any other material typically employed to hold reagents or food; envelopes, that may include foil-lined interiors, such as aluminum foil or an alloy. Other containers include test tubes, vials, flasks, and syringes. The containers may have two compartments that are separated by a readily removable membrane that upon removal permits the io components to mix. Removable membranes may be glass, plastic, rubber, or the like.

Kits may also include instruction materials. Instructions may be printed on paper or other substrates, and/or may be supplied as an electronic-readable medium, such as a floppy disc, CD-ROM, DVD-ROM, Zip disc, videotape, audio tape, etc. Detailed instructions may not be physically associated with the kit; instead, a user may be directed to an internet web site specified by the manufacturer or distributor of the kit, or supplied as electronic mail.

An embodiment of a kit of the invention would include a stimulant laxative; an osmotic purgative; and instructions for use of the kit comprising directions to consume an effective amount of the stimulant laxative 3 days and 2 days prior to the day for which maximum cleansing is desired, and to consume an effective amount, in split doses, of the osmotic purgative 1 day prior to the day for which maximum cleansing is desired. Alternate instructions for use of the kit would direct subjects to consume an effective amount of the stimulant laxative at least 2 days prior to the day for which maximum cleansing is desired, and to consume an effective amount of the osmotic purgative 1 day prior to the day for which maximum cleansing is desired. Another kit may have instructions for use of the kit comprising directions to consume an effective amount of the stimulant laxative 4, 3, and 2 days prior to the day for which maximum cleansing is desired, and to consume an effective amount of the osmotic purgative 1 day prior to the day for which maximum cleansing is desired. Kits of the invention may include Gatorade®. Osmotic purgative in the kits may be one or more of magnesium citrate, Oral Sodium Phosphate, PICO-SALAX®, CITRO-MAG™ Stimulant laxative in the kits may be one or more of sodium picosulphate, senna, bisacodyl, or a combination thereof.

In summary, stimulant laxative taken on days leading up to, but not on the day of consumption of osmotic purgative have been shown to provide enhanced efficacy particularly in the right colon, while maintaining excellent patient tolerability and safety.

Embodiments of the invention are further described by way of the following non-limiting examples.

### WORKING EXAMPLES

### Example 1

### Parameters of Studies

Studies were conducted to compare three main pre-colonoscopy colon cleansing regimens. Goals of these studies included evaluating efficacy, patient tolerance, and patient safety of these regimens. In total, three hundred and fifty-one patients were enrolled into Groups 1A, 2, and 3. Thirty-two patients of the three hundred and fifty-one were randomized but never participated (specifically, 9 from Group 1A, 13 from Group 2, and 10 from Group 3) because n=95 in each group had been reached. An additional fifteen patients (n=5 in each of Group 1A, 2, and 3) completed their cleansing preparations but had incomplete colonoscopies for reasons other than preparation, hence preventing completion of the OBPS. Safety and patient tolerance data on these fifteen patients were included in the analysis. Colon cleansing data was collected for 100 patients from Group 1A, 104 patients from Group 2, and 96 patients from Group 3. There were no significant differences among the groups in baseline characteristics including age, weight, gender, or baseline biochemistry (see Table I)

These studies were prospective, randomized, and single-blinded, such that the endoscopy team was unaware of the assigned treatment Group for each patient. Investigator blinding was maintained by a research assistant and through repeated instructions to patients not to divulge their cleansing regimen to attending endoscopy unit personnel.

Patients gave informed written consent prior to enrollment and studies were approved by the Queen's University at Kingston's (Kingston, Ontario, Canada) to human ethics committee. Adult patients undergoing outpatient colonoscopy had been invited to participate in the studies. As each treatment regimen has a low volume osmotic purgative solution and hence has the potential to induce fluid shifts, patients at risk for adverse events were excluded. Adverse events include, for example, renal impairment (creatinine>normal range for age and gender), recent myocardial infarction or angina, and ascites. Patients who had colorectal resection previously and those who were pregnant were also excluded.

Randomization was conducted in random size permuted blocks using a computer generated table prepared by an independent biostatistician. Group assignments were revealed to each patient and to a research assistant by their opening an opaque envelope together, after informed consent had been obtained. When the Group assignment was known to the patient and research assistant, the patient was provided with a copy of the appropriate instruction sheet (see FIGS. 4A-C) and appropriate laxatives.

### Example 2

### Pre-Colonoscopy Colon Cleansing

All patients were instructed to ingest only clear fluids on the day prior to colonoscopy. They were encouraged to drink four litres of Gatorade® or similar fluids the evening prior to colonoscopy. Patients were randomly assigned to Group 1A, Group 2, or Group 3. Another smaller study of patients in Group 1B (n=12) was performed separately.

Patients in Group 1A were provided with the appropriate instruction sheet (see FIG. 4A) and appropriate products. Briefly, patients of Group 1A were directed to consume (1) 10 mg bisacodyl orally at 5 pm three days prior to colonoscopy, (2) 10 mg bisacodyl orally at 5 pm two days prior to colonoscopy, (3) 1 sachet of PICO-SALAX® at 5 pm one day prior to colonoscopy, and (4) a second sachet of PICO-SALAX® at 10 pm one day prior to colonoscopy.

Patients in Group 1B were provided with the appropriate instruction sheet (see FIG. 4D) and appropriate products. Briefly, patients of Group 1B were directed to consume (1) 10 mg bisacodyl orally at 5 pm two days prior to colonoscopy, (2) 1 sachet of PICO-SALAX® at 5 pm one day prior to colonoscopy, and (3) a second sachet of PICO-SALAX® at about 10 pm one day prior to colonoscopy.

Patients in Group 2 were provided with the appropriate instruction sheet (see FIG. 4B) and appropriate product. Briefly, patients of Group 2 were directed to consume: (1) 1 sachet of PICO-SALAX® at 5 pm one day prior to colonoscopy and (2) a second sachet at 10 pm one day prior to colonoscopy.

Patients in Group 3 were provided with the appropriate instruction sheet (see FIG. 4C) and appropriate product. Briefly, instructions provided to Group 3 directed patients to consume (1) 45 mL of Oral Sodium Phosphate at 5 pm one day prior to colonoscopy and (2) a second 45 mL at 10 pm one day prior to colonoscopy.

### Example 3

### Patient Acceptance/Tolerability

On the morning of colonoscopy and prior to the procedure, patients completed a rating scale questionnaire (see example in FIG. 5) designed to assess their tolerance of the colonic cleansing regimen. By completing the questionnaire, patients reported their evaluation of whichever colon cleansing regime they experienced. Feedback parameters included its tolerability, taste, and whether they experienced nausea, vomiting, abdominal pain, chest pain, dizziness, numbness/tingling, bloating. Patients also compared their experience to any other colon cleansing regime that they had experienced in their past. Results are presented graphically in FIGS. 2A-B, and 3A-B.

### Example 4

### Endoscopic Evaluation

Quality of colonic cleansing was assessed using a previously validated system called the Ottawa Bowel Prep Scale (see example in FIG. 6). The Ottawa Bowel Prep Scale was completed by an attending gastroenterologist in the outpatient endoscopy unit at Hotel Dieu Hospital, Kingston, Ontario, Canada. Endoscopists were instructed not to ask patients about the details of their particular pre-colonoscopy colonic cleansing regime. Colon cleanliness was scored at the end of each colonoscopy. Endoscopists quantified the cleanliness, that is, they rated the quality of visualization of the colon lining as follows:
(0) Excellent: Mucosal detail clearly visible. If fluid present, it is clear. Almost no stool residue.
(1) Good: Some turbid fluid or stool residue but mucosal detail still visible. Washing and suctioning not necessary.
(2) Fair: Turbid fluid or stool residue obscuring mucosal detail and contour. However, mucosal detail becomes visible with suctioning. Washing not necessary.
(3) Poor: Presence of stool obscuring mucosal detail and contour. However, with suctioning and washing, a reasonable view is obtained.
(4) Inadequate: Solid stool obscuring mucosal detail and contour despite aggressive washing and suctioning.
   Thus, a low score such as zero or one indicates excellent cleanliness. FIGS. 1A and 1B indicate the percentage of patients from specified Groups with respective colon sections scored as 4 “Inappropriate”, or 3 “Poor”.

### Example 5

### Statistical Analysis

A primary endpoint for these studies was cleansing efficacy. Previous data using the OBPS demonstrated a normal distribution with a mean standard deviation of 4.3 points. A two-point average difference between Groups 1A, 2, and 3 was considered minimally clinically significant. With a standard deviation of 4.3 and to targeted 95 subjects per group for Groups 1A, 2, and 3, one way ANOVA testing would provide 80% power to detect a between-group difference of 2 points with a two-sided alpha of 0.05. To account for an expected combined drop-out/incomplete colonoscopy rate of 15%, approximately 115 patients were enrolled in each group of Groups 1A, 2, and 3. The target of having colonoscopies completed for at least 95 patients in each Group was met. Total OBPS was analyzed as a continuous variable, after confirmation of normal distribution, using one way ANOVA tests. Tolerability and efficacy in the different components of the OBPS (i.e. right, middle, and left colon) were analyzed using the Mantel-Haenszel trend test. Other variables are described as mean±standard deviation and compared using one way ANOVA, or described as counts and percentages and compared using the Chi squared test. Analysis of secondary endpoints was corrected by Bonferroni, with an adjusted significance of P value=0.01. Gender and time of colonoscopy were analyzed post-hoc.

Although this invention is described in detail with reference to preferred embodiments thereof, these embodiments are offered to illustrate but not to limit the invention. It is possible to make other embodiments that employ the principles of the invention and that fall within its spirit and scope as defined by the claims appended hereto.

TABLE I
Summary of baseline characteristics of patients In each group

| | Sodium Phosphate | Picosalax | Picosalax plus | P |
|---|---|---|---|---|
| | n = 101 | n = 109 | bisacodyl n = 105 | value |
| Mean Age ± Standard Deviation | 55.6 ± 9.5 | 54.9 ± 10.1 | 54.4 ± 9.5 | 0.66 |
| Female | 54.4% | 47.8% | 51.4% | 0.62 |
| Weight | 78.9 ± 15.7 kg | 81.5 ± 16.9 kg | 83.0 ± 17.8 kg | 0.21 |
| Hypertension | 21.8% | 24.8% | 23.1% | 0.88 |
| Diabetes | 2% | 2.8% | 3.8% | 0.9 |
| Diuretic use | 6.9% | 12.8% | 15.4% | 0.16 |
| ACE inhibitor use | 7.9% | 13.8% | 13.5% | 0.34 |

TABLE II
Comparison of hemodynamic and biochemical data measured at baseline and morning of colonoscopy

| | Sodium Phosphate | | Picosalax | | Picosalax plus | | |
|---|---|---|---|---|---|---|---|
| | n = 101 | | n = 107 | | bisacodyl n = 105 | | P value |
| Δweight | −0.7 ± 3.3 | kg | −0.9 ± 3 | kg | −1 ± 3 | kg | 0.83 |

| | | | | | |
|---|---|---|---|---|---|
| Postural | ΔΔsystolic BP | 2.3 ± 4.8 | 0.1 ± 17.3 | 1.8 ± 15.8 | 0.57 |
| vitals | ΔΔDiastolic BP | 3.1 ± 10.2 | 0 ± 10.5 | 1.8 ± 9.7 | 0.09 |
| signs | ΔΔPulse | 2.3 ± 10.2 | 3 ± 9.4 | 5 ± 8.3 | 0.09 |

| ΔHemoglobin | 0.3 ± 6.3 | g/dL | 3.4 ± 6.4 | g/dL | 3.3 ± 7 | g/dL | 0.001 |
|---|---|---|---|---|---|---|---|
| Δhematocrit | 0 ± 0.02 | | 0.01 ± 0.02 | | 0.01 ± 0.02 | | 0.001 |
| ΔSodium | 0.6 ± 2.6 | mmol/L | −1.1 ± 2.6 | mmol/L | −1.1 ± 2.5 | mmol/L | <0.001 |
| Δurea | −2.3 ± 1.2 | mmol/L | −2.1 ± 1.1 | mmol/L | −2.4 ± 1.3 | mmol/L | 0.26 |
| Δcreatinine | 0.7 ± 6.8 | umol/L | −1.4 ± 10.1 | umol/L | 0 ± 2.3 | umol/L | 0.16 |
| Δestimated GFR | −1 ± 8.5 | mL/min | 0.2 ± 9.8 | mL/min | 0.5 ± 9.4 | mL/min | 0.48 |
| Δcalcium | −0.1 ± 0.1 | mmol/L | 0 ± 0.1 | mmol/L | 0 ± 0.2 | mmol/L | <0.001 |
| % below normal range | 21.7 | | 1.8 | | 2.9 | | <0.001 |
| ΔChloride | −0.6 ± 2.1 | mmol/L | −2 ± 2.5 | mmol/L | −1.2 ± 2.7 | mmol/L | <0.001 |
| ΔPotassium | −0.6 ± 2.1 | mmol/L | −0.2 ± 0.4 | mmol/L | −0.2 ± 0.4 | mmol/L | <0.001 |
| % below normal range | 72.5 | | 17.5 | | 10 | | <0.001 |
| ΔMagnesium | 0 ± 0.1 | mmol/L | 0.1 ± 0.1 | mmol/L | 0.1 ± 0.1 | mmol/L | <0.001 |
| % above normal range | 0 | | 39.8 | | 32.4 | | <0.001 |
| ΔPhosphate | 0.4 ± 0.3 | mmol/L | 0 ± 0.2 | mmol/L | −0.1 ± 0.2 | mmol/L | <0.001 |

| | | | | |
|---|---|---|---|---|
| % above normal range | 47.5 | 2.8 | 0 | <0.001 |

TABLE III
Summary of Patient Diary Information for Laxative Use

| | Mean time to | Mean time to | |
|---|---|---|---|
| | first BM ± | last BM ± | Total number |
| | standard | standard | BM ± standard |
| | deviation (h) | deviation (h) | deviation |
| Bisacodyl (10 mg) | 8.9 ± 5.1 | 16.5 ± 5.1 | 3.4 ± 2 |
| taken 3 nights before | | | |
| colonoscopy (e.g, at | | | |
| suppertime) | | | |
| Bisacodyl (10 mg) | 8.6 ± 5.3 | 16.5 ± 5.3 | 3.8 ± 2 |
| taken 2 nights before | | | |
| colonoscopy (e.g, at | | | |
| suppertime) | | | |
| PICOSALAX (1^{st} of 2 | 1.5 ± 1.3 | 5.4 ± 2.5 | 4.4 ± 2.7 |
| parts of a split dose) | | | |
| taken evening before | | | |
| colonoscopy (e.g. at 5 | | | |
| pm) | | | |
| PICOSALAX (2^{nd} of 2 | 1.25 ± 1.3 | 9.6 ± 4.6 | 6.5 ± 3.2 |
| parts of a split dose) | | | |
| taken evening before | | | |
| colonoscopy (e.g. at 10 | | | |
| pm) | | | |
| PICOSALAX (2^{nd} of 2 | 0.9 ± 0.8 | 3.85 ± 1 | 4.5 ± 2.4 |
| parts of a split dose) | | | |
| taken morning of | | | |
| colonoscopy (e.g. at 7 | | | |
| am) | | | |

BM = bowel movements

### REFERENCES

Afridi, S. A.; Barthel, J. S.; King, P. D.; Pineda, J. J.; Marshall, J. B. (1995). “Prospective, randomized trial comparing a new sodium phosphate-bisacodyl regimen with conventional PEG-ES lavage for outpatient colonoscopy”. Gastrointest. Endos.41(5): 485-489.
Barclay R. L.; Depew W. T.; & Vanner S. J. (2002). “Carbohydrate-electrolyte rehydration protects against intravascular volume contraction during colonic cleansing with orally administered sodium phosphate”. Gastrointest. Endosc. 56: 633-638.
Belsey, J.; Epstein, O.; Heresbach, D. (2007). “Systematic review: oral bowel preparation for colonoscopy”. Aliment. Pharmacol. Ther. 25: 373-384.
Brooker, J. C.; Saunders, B. P.; Shah, S. G.; Thapar, C. J.; Thomas, H. J.; Atkin, W. S.; Cardwell, C. R.; Williams, C. B. (2002). “Total colonic dye-spray increases the detection of diminutive adenomas during routine colonoscopy: a randomized controlled trial”. Gastrointest. Endosc. 56: 333-338.
Chiu, H. M.; Lin, J. T.; Wang, H. P.; Lee, Y. C.; Wu, M. S. (2006). “The impact of colon preparation timing on colonoscopic detection of colorectal neoplasms—a prospective endoscopist-blinded randomized trial”. Am. J. Gastroenterol. 101: 2719-2725.
Desmeules, S.; Bergeron, M. J.; Isenring, P. (2003). “Acute phosphate nephropathy and renal failure”. N. Engl. J. Med. 349: 1006-1007.
Ell, C.; Fischbach, W.; Keller, R.; Dehe, M.; Mayer, G.; Schneider, B.; Albrecht, U.; Schuette, W. (2003). “A randomized, blinded, prospective trial to compare the safety and efficacy of three bowel-cleansing solutions for colonoscopy (HSG-01*)”. Endoscopy 35: 300-304.
Froehlich, F.; Wietlisbach, V.; Gonvers, J. J.; Burnand, B.; Vader, J. P. (2005). “Impact of colonic cleansing on quality and diagnostic yield of colonoscopy: the European Panel of Appropriateness of Gastrointestinal Endoscopy European multicenter study”. Gastrointest. Endosc. 61: 378-384.
Gupta, T.; Mandot, A.; Desai, D.; Abraham, P.; Joshi, A.; Shah, S. (2007). “Comparison of two schedules (previous evening versus same morning) of bowel preparation for colonoscopy”. Endoscopy 39: 706-709.
Hookey, L. C.; Depew, W. T.; Vanner, S. (2002). “The safety profile of oral sodium phosphate for colonic cleansing before colonoscopy in adults”. Gastrointest. Endosc. 56: 895-902.
Hookey, L. C.; Depew, W. T.; Vanner, S. (2004). “A prospective randomized trial comparing low-dose oral sodium phosphate plus stimulant laxatives with large volume polyethylene glycol solution for colon cleansing”. Am. J. Gastro. 2217-2222.
Hookey. L. C.; Vanner, S. (2007). “A review of current issues underlying colon cleansing before colonoscopy”. Can. J. Gastroenterol. 21: 105-111.
Hsu, C. W.; Imperiale, T. F. (1998). “Meta-analysis and cost comparison of polyethylene glycol lavage versus sodium phosphate for colonoscopy preparation”. Gastrointest. Endosc. 48: 276-282.
Markowitz, G. S.; Nasr, S. H.; Klein, P.; Anderson, H.; Stack, J. I.; Alterman, L.; Price, B.; Radhakrishnan, J.; D'Agati, V. D. (2004). “Renal failure due to acute nephrocalcinosis following oral sodium phosphate bowel cleansing”. Hum. Pathol. 35: 675-684.
Markowitz, G. S.; Radhakrishnan, J.; D'Agati, V. D. (2007). “Towards the incidence of acute phosphate nephropathy”. J. Am. Soc. Nephrol. 18: 3020-3022.
Markowitz, G. S.; Stokes, M. B.; Radhakrishnan, J.; D'Agati, V. D. (2005b). “Acute phosphate nephropathy following oral sodium phosphate bowel purgative: an underrecognized cause of chronic renal failure”. J. Am. Soc. Nephrol. 16: 3389-3396.
Markowitz, G. S.; Whelan, J.; D'Agati, V. D. (2005c). “Renal failure following bowel cleansing with a sodium phosphate purgative”. Nephrol. Dial. Transplant 20: 850-851.
Okamoto, M.; Kawabe, T.; Yamaji, Y.; Kato, J.; Ikenoue, T.; Togo, G.; Watabe, H.; Yoshida, H.; Shiratori, Y.; Omata, M. (2005) “Flat-type early colorectal cancer preferentially develops in right-sided colon in older patients”. Dis. Colon Rectum 48(1): 101-107.
Park, D. H.; Kim, H. S.; Kim, W. H.; Kim, T. I.; Kim, Y. H.; Park, D. I.; Kim, H. J.; Yang, S. K.; Byeon, J. S.; Lee, M. S.; Chung, I. K.; Jung, S. A.; Jeen, Y. T.; Choi, J.H.; Choi, H.; Han, D. S. (2008). “Clinicopathologic characteristics and malignant potential of colorectal flat neoplasia compared with that of polypoid neoplasia”. Diseases of the Colon and Rectum 51: 43-49.
Park, J. S.; Sohn, C. I.; Hwang, S. J.; Choi, H. S.; Park, J. H.; Kim, H. J.; Park, D. I.; Cho, Y. K.; Jeon, W. K.; Kim, B. I. (2007). “Quality and effect of single dose versus split dose of polyethylene glycol bowel preparation for early-morning colonoscopy”. Endoscopy 39: 616-619.
Rex, D. K.; Imperiale, T. F.; Latinovich, D. R.; Bratcher, L. L. (2002). “Impact of bowel preparation on efficiency and cost of colonsocopy”. Am. J. Gastroenterol. 97: 1696-1700.
Rex, D. K. (2006). “Maximizing detection of adenomas and cancers during colonoscopy”. Am. J. Gastroenterol. 101: 2866-2877.
Rex, D. K. (2007). “Dosing considerations in the use of sodium phosphate bowel preparations for colonoscopy”. Ann. Pharmacother. 41: 1466-1475.
Rostom, A.; Jolicoeur, E. (2004). “Validation of a new scale for the assessment of bowel preparation quality”. Gastrointest. Endosc. 59: 482-486.
Rostom, A.; Jolicoeur, E.; Dube, C.; Gregoire, S.; Patel, D.; Saloojee, N.; Lowe, C. (2006). “A randomized prospective trial comparing different regimens of oral sodium phosphate and polyethylene glycol-based lavage solution in the preparation of patients for colonoscopy”. Gastrointest. Endosc. 64: 544-552.
Saitoh, Y.; Waxman, I.; West, A. B.; Popnikolov, N. K.; Gatalica, Z.; Watari, J.; Obara, T.; Kohgo, Y.; Pasricha, P. J. (2001). “Prevalence and distinctive biologic features of flat colorectal adenomas in a North American population”. Gastroenterology 120: 1657-1665.
Tan, J. J.; Tjandra, J. J. (2006). “Which is the optimal bowel preparation for colonoscopy—a meta-analysis”. Colorectal. Dis. 8: 247-258.
Vanner, S. J.; MacDonald, P. H.; Paterson, W. G.; Prentice, R. S.; Da Costa, L. R.; Beck, I. T. (1990). “A randomized prospective trial comparing oral sodium phosphate with standard polyethylene glycol-based lavage solution (Golytely) in the preparation of patients for colonoscopy”. Am. J. Gastroenterol. 85: 422-427.

1. A method of colon cleansing comprising:
consuming an effective amount of a stimulant laxative 3 days and 2 days prior to the day for which maximum cleansing is desired;
drinking fluids and restricting solid food for one day prior to the day for which maximum cleansing is desired; and
consuming an effective amount of an osmotic purgative one day prior to the day for which maximum cleansing is desired.

2. A method of colon cleansing comprising:
consuming an effective amount of a stimulant laxative at least 2 days prior to the day for which maximum cleansing is desired;
drinking fluids and restricting solid food for one day prior to the day for which maximum cleansing is desired; and
consuming an effective amount of an osmotic purgative one day prior to the day for which maximum cleansing is desired.

3. A method of colon cleansing comprising:
consuming an effective amount of a stimulant laxative 4, 3, and 2 days prior to the day for which maximum cleansing is desired;
drinking fluids and restricting solid food for one day prior to the day for which maximum cleansing is desired; and
consuming an effective amount of an osmotic purgative one day prior to the day for which maximum cleansing is desired.

4. The method of claim 1, wherein drinking fluids comprises consuming an effective amount of rehydration fluid one day prior to the day for which maximum cleansing is desired.

5. The method of claim 4, wherein the rehydration fluid is Gatorade®.

6. The method of claim 4, wherein the effective amount of rehydration fluid is about 3 to about 5L.

7. The method of claim 1, wherein the osmotic purgative comprises PICO-SALAX®.

8. The method of claim 1, wherein the osmotic purgative comprises a small volume osmotic purgative which is Oral Sodium Phosphate, magnesium citrate, CITRO-MAG™, PICO-SALAX®, or a combination thereof.

9. The method of claim 1, or wherein the osmotic purgative is consumed in split doses.

10. The method of claim 9, wherein the split doses are consumed about twelve hours apart.

11. The method of claim 10, wherein the split doses are consumed about four to about six hours apart.

12. The method of claim 10, wherein the split doses are consumed about five hours apart.

13. The method of claim 1, wherein the stimulant laxative comprises one or more of bisacodyl, senna, and sodium picosulphate.

14. The method of claim 1, wherein the stimulant laxative comprises bisacodyl and the osmotic purgative comprises magnesium citrate.

15. The method of claim 1, wherein the stimulant laxative comprises sodium picosulphate and the osmotic purgative comprises magnesium citrate.

16. The method of claim 14, wherein drinking fluids comprises consuming an effective amount of rehydration fluid one day prior to the day for which maximum cleansing is desired.

17. A kit for colon cleansing comprising:
a stimulant laxative;
an osmotic purgative; and
instructions for use of the kit comprising directions to consume an effective amount of the stimulant laxative 3 days and 2 days prior to the day for which maximum cleansing is desired, and to consume an effective amount of the osmotic purgative 1 day prior to the day for which maximum cleansing is desired.

18. A kit for colon cleansing comprising:
a stimulant laxative;
an osmotic purgative; and
instructions for use of the kit comprising directions to consume an effective amount of the stimulant laxative at least 2 days prior to the day for which maximum cleansing is desired, and to consume an effective amount of the osmotic purgative 1 day prior to the day for which maximum cleansing is desired.

19. A kit for colon cleansing comprising:
a stimulant laxative;
an osmotic purgative; and
instructions for use of the kit comprising directions to consume an effective amount of the stimulant laxative 4, 3, and 2 days prior to the day for which maximum cleansing is desired, and to consume an effective amount of the osmotic purgative 1 day prior to the day for which maximum cleansing is desired.

20. The kit of claim 17, wherein the stimulant laxative is sodium picosulphate, senna, bisacodyl, or a combination thereof.

21. The kit of claim 17, wherein the osmotic purgative is PICO-SALAX®.

22. The kit of claim 17, wherein the osmotic purgative is Oral Sodium Phosphate, magnesium citrate, PICO-SALAX®, CITRO-MAG™, or a combination thereof.

23. The kit of claim 17, further comprising rehydration fluid.
